(19) United States
(12) Patent Application Publication
(10) Pub. No.: US 2023/0340589 A1
(43) Pub. Date: Oct. 26, 2023

(54) MAGNETIC SENSOR ARRAYS FOR NUCLEIC ACID SEQUENCING AND METHODS OF MAKING AND USING THEM

(71) Applicants: Western Digital Technologies, Inc., San Jose, CA (US); Roche Sequencing Solutions, Inc., Pleasanton, CA (US)

(72) Inventors: Patrick BRAGANCA, San Jose, CA (US); Neil Charles SMITH, San Jose, CA (US); Juraj TOPOLANCIK, Redwood City, CA (US); Yann ASTIER, Pleasanton, CA (US)

(73) Assignees: Western Digital Technologies, Inc., San Jose, CA (US); Roche Sequencing Solutions, Inc., Pleasanton, CA (US)

(21) Appl. No.: 17/905,724

(22) PCT Filed: Mar. 7, 2021
(86) PCT No.: PCT/US21/21274

§ 371 Date: Sep. 6, 2022

Related U.S. Application Data
(60) Provisional application No. 62/987,831, filed on Mar. 10, 2020.

(51) Int. Cl. C12Q 1/6874; B01L 3/00

(52) U.S. Cl. CPC C12Q 1/6874; B01L 3/5085; B01L 2300/0663; B01L 2300/12

(57) ABSTRACT

Disclosed herein are apparatuses for nucleic acid sequencing using magnetic labels (e.g., magnetic particles) and magnetic sensors. Also disclosed are methods of making and using such apparatuses. An apparatus for nucleic acid sequencing comprises a plurality of magnetic sensors, a plurality of binding areas disposed above the plurality of magnetic sensors, each of the binding areas for holding fluid, and at least one line for detecting a characteristic of at least a first magnetic sensor of the plurality of magnetic sensors, the characteristic indicating presence or absence of one or more magnetic nanoparticles coupled to a first binding area associated with the first magnetic sensor.

### CROSS-REFERENCE TO RELATED APPLICATIONS

This application claims priority to, and hereby incorporates by reference in its entirety the contents of, U.S. provisional application No. 62/987,831, filed Mar. 10, 2020 and entitled “MAGNETIC SENSOR ARRAYS FOR NUCLEIC ACID SEQUENCING AND METHODS OF MAKING AND USING THEM” (Attorney Docket No. ROA-1001P-US/P35967-US).

### BACKGROUND

Sequencing by synthesis (SBS) has been a successful commercially-viable method to obtain large quantities of DNA sequencing data. SBS involves binding of primer-hybridized template DNA, incorporation of a deoxynucleoside triphosphate (dNTP), and detection of incorporated dNTP.

Current sequencing systems use fluorescence signal detection. Four fluorescently-labeled nucleotides are used to sequence millions of clusters in parallel. DNA polymerase catalyzes the incorporation of fluorescently-labeled dNTPs into a DNA template strand during sequential cycles of DNA synthesis. During each cycle, a single labeled dNTP is added to the nucleic acid chain. The nucleotide label serves as a “reversible terminator” for polymerization. After the dNTP has been incorporated, the fluorescent dye is identified through laser excitation and imaging, then enzymatically cleaved to allow the next round of incorporation. The base is identified directly from signal intensity measurements during each cycle.

State-of-the-art sequencing systems that rely on fluorescence signal detection can provide throughputs of up to 20 billion reads per run. Achieving such performance, however, requires large-area flow cells, high-precision free-space imaging optics, and expensive high-power lasers to generate sufficient fluorescence signals to enable successful base detection.

Two general strategies have enabled a gradual increase in SBS throughput (e.g., characterized by base reads per run). The first approach has been outward scaling, by increasing the size and the number of flow-cells in the sequencers. This approach increases both the cost of reagents and the price of the sequencing system, because it requires additional high-power lasers and high-precision nano-positioners.

The second approach involves inward scaling, where the size of individual DNA testing sites is reduced so that the number of sequenced DNA strands in a fixed-size flow-cell is higher. This second approach is more appealing to reduce the overall sequencing cost because additional cost only involves implementation of better imaging optics while keeping the cost of consumables the same. But higher numerical aperture (NA) lenses must be employed to distinguish the signal from neighboring fluorophores. This approach has limits because the Rayleigh criterion puts the distance between resolvable light point sources at 0.61 λ/NA, i.e., even in advanced optical imaging systems, the minimum distance between two sequenced DNA strands cannot be reduced beyond approximately 400 nm. Similar resolution limits apply to sequencing directly on top of imaging arrays where the smallest pixel size achieved so far is less than 1 μm. The Rayleigh criterion currently represents the fundamental limitation for inward scaling of optical SBS systems. Overcoming these limitations may require super-resolution imaging techniques, which has not yet been achieved in highly multiplexed systems. Hence, at this stage, the only practicable way to increase the throughput of optical SBS sequencers is to build bigger flow-cells and more expensive optical scanning and imaging systems.

Thus, there is a need to improve SBS.

### BRIEF DESCRIPTION OF THE DRAWINGS

Objects, features, and advantages of the disclosure will be readily apparent from the following description of certain embodiments taken in conjunction with the accompanying drawings in which:

FIG. 1 illustrates a portion of a magnetic sensor in accordance with some embodiments.

FIGS. 2A and 2B illustrate the resistance of magnetoresistive (MR) sensors in accordance with some embodiments.

FIG. 3A illustrates the concept of using a spin torque oscillator (STO) sensor in accordance with some embodiments.

FIG. 3B shows the experimental response of a STO through a delay detection circuit when an AC magnetic field is applied across the STO in accordance with some embodiments.

FIGS. 3C and 3D illustrate how STOs may be used as nanoscale magnetic field detectors in accordance with some embodiments.

FIG. 4A is a top view of a portion of a sequencing apparatus in accordance with some embodiments.

FIGS. 4B and 4C are cross-section views of the portion of the sequencing apparatus shown in FIG. 4A.

FIG. 4D is a block diagram showing components of the apparatus of FIGS. 4A, 4B, and 4C in accordance with some embodiments.

FIGS. 5A and 5B illustrate two approaches to selecting magnetic sensors in accordance with some embodiments.

FIG. 6 illustrates a method of manufacturing a sequencing apparatus in accordance with some embodiments.

FIG. 7 illustrates a method of using the sequencing apparatus for nucleic acid sequencing in accordance with some embodiments.

FIG. 8 illustrates a method of using the sequencing apparatus in which multiple nucleotide precursors are introduced substantially simultaneously in accordance with some embodiments.

To facilitate understanding, identical reference numerals have been used, where possible, to designate identical elements that are common to the figures. It is contemplated that elements disclosed in one embodiment may be beneficially utilized in other embodiments without specific recitation. Moreover, the description of an element in the context of one drawing is applicable to other drawings illustrating that element.

### DETAILED DESCRIPTION

Disclosed herein are apparatuses for nucleic acid sequencing using magnetic labels (e.g., magnetic particles) and magnetic sensors. Also disclosed are methods of making and using such apparatuses. For simplicity, some of the discussions below refer to sequencing DNA as an example. It is to be understood that the disclosures herein apply generally to nucleic acid sequencing.

The inventors recognized that the resolution limits of fluorescence microscopy and CMOS imagers, as used in prior art SBS, do not apply to electric charge (e.g., silicon nanowire field-effect transistors (FETs)) or magnetic field sensors (e.g., spin valves, magnetic tunnel junctions (MTJs), spin-torque oscillators (STOs), etc.), where the size of sensing elements is an order of magnitude smaller and the level of multiplexing considerably higher than in state-of-the-art SBS systems. Magnetic field sensing in SBS is particularly appealing because DNA and sequencing reagents are non-magnetic, which enables significant improvements to signal-to-noise ratio (SNR) compared to electric charge sensing schemes based on electron transport modulation in CMOS components. Furthermore, magnetic sensing does not require the incorporated bases to be in direct contact with the junction. Miniaturized magnetic field sensors can be used to detect nanoscale magnetic nanoparticles to perform SBS.

Performing SBS using magnetic sensor arrays can dramatically increase the throughput and reduce the cost of sequencing by providing additional inward scaling by a factor of, for example, approximately 100 while eliminating the need for high-power lasers and high-resolution optics in sequencing systems.

This document discloses SBS protocols that use magnetically-labeled nucleotide precursors in conjunction with sequencing devices that include arrays of magnetic sensing elements (e.g., MTJs, STOs, spin valves, etc.). The devices also include one or more etched binding areas that allow the magnetic sensors to detect the magnetic labels in the magnetically-labeled nucleotide precursors while protecting the magnetic sensors from damage (e.g., using a thin layer of insulator).

Among the disclosures herein is the disclosure of an apparatus for nucleic acid sequencing, the apparatus comprising a plurality of magnetic sensors, a plurality of binding areas disposed above the plurality of magnetic sensors, each of the binding areas for holding fluid, and at least one line for detecting a characteristic of at least a first magnetic sensor of the plurality of magnetic sensors, the characteristic indicating presence or absence of one or more magnetic nanoparticles coupled to a first binding area associated with the first magnetic sensor. In some embodiments, the first magnetic sensor comprises a magnetoresistive (MR) device. The MR device may comprise a pinned layer, a free layer, and a barrier layer disposed between the pinned layer and the free layer. In some such embodiments, in the absence of the one or more magnetic nanoparticles coupled to the first binding area, a magnetic moment of the pinned layer is approximately 90 degrees from a magnetic moment of the free layer.

The first binding area may include a structure (e.g., a cavity or ridge) configured to anchor nucleic acid to the first binding area.

In some embodiments, the shape of the first magnetic sensor is substantially cylindrical or substantially cuboid. In some embodiments, a lateral dimension of the first magnetic sensor is between approximately 10 nanometer (nm) and approximately 1 micrometer.

The apparatus may also include sensing circuitry coupled to the plurality of magnetic sensors via the at least one line. The sensing circuitry may be configured to apply a current to the at least one line to detect the characteristic (e.g., a magnetic field, a resistance, a change in magnetic field, a change in resistance, a noise level, etc.) of the first magnetic sensor. In some embodiments, the sensing circuitry comprises a magnetic oscillator, and the characteristic is a frequency of a signal associated with or generated by the magnetic oscillator.

The apparatus may have an insulating material (e.g., an oxide (e.g., silicon dioxide, aluminum oxide, etc.), a nitride (e.g., silicon nitride, etc.)) disposed between the plurality of magnetic sensors and the plurality of binding areas. The thickness of the insulating material between a top of the first magnetic sensor and the first binding area may be, for example, between approximately 3 nm and approximately 20 nm.

In some embodiments, the at least one line includes a first line disposed above a top surface of the first magnetic sensor, and the first binding area is located within a trench in the first line, the trench being above the top surface of the first magnetic sensor.

In some embodiments, the plurality of magnetic sensors is arranged in a rectangular array, and the at least one line includes at least a first line and a second line, the first line being disposed above the first magnetic sensor and the second line being disposed below the first magnetic sensor. One or more binding areas may be located within trenches in the first line. In some embodiments, the first line is coupled to a row of the rectangular array and the second line is coupled to a column of the rectangular array, or vice versa.

Also disclosed herein are methods of manufacturing apparatuses for nucleic acid sequencing. In some embodiments, a method of manufacturing a nucleic acid sequencing device comprises fabricating a first line, fabricating a plurality of magnetic sensors, depositing insulating material between the magnetic sensors, fabricating a plurality of additional lines, and creating a plurality of binding areas. In some embodiments, each magnetic sensor's bottom surface is coupled to the first line, and each magnetic sensor's top surface is coupled to a respective one of the additional lines.

Fabricating the first line may comprise depositing a metal layer on a substrate (e.g., using physical vapor deposition, ion beam deposition, etc.), and patterning the metal layer into the first line (e.g., using photolithography, milling, and/or etching).

In some embodiments, after fabricating the first line and before fabricating the plurality of magnetic sensors, insulating material is deposited over the first line, the first line is then uncovered (e.g., using chemical mechanical polishing (CMP)), and the plurality of magnetic sensors is fabricated on the uncovered first line.

The plurality of magnetic sensors may be fabricated by depositing a plurality of layers on the first line, and patterning the plurality of layers (e.g., using photolithography and/or etching) to form the plurality of magnetic sensors, each of the plurality of magnetic sensors having a predetermined shape (e.g., substantially cylindrical, substantially cuboid, etc.). Depositing the plurality of layers may comprise depositing a first ferromagnetic layer, depositing a metal or insulator layer over the first ferromagnetic layer, and depositing a second ferromagnetic layer over the metal or insulator layer. A lateral dimension of each of the plurality of magnetic sensors may be, for example, between approximately 10 nm and approximately 1 micrometer.

In some embodiments, the plurality of magnetic sensors is in a rectangular array, and the first line corresponds to a row of the rectangular array, and each of the plurality of additional lines corresponds to a column of the rectangular array, or vice versa.

In some embodiments, after depositing the insulating material between the magnetic sensors and before fabricating the plurality of additional lines, a chemical mechanical polishing step is performed to expose the top surface of each of the plurality of magnetic sensors.

In some embodiments, fabricating the plurality of additional lines comprises depositing a layer of metal, performing photolithography to define the plurality of additional lines, and removing a portion of the layer of metal.

In some embodiments, creating the plurality of binding areas comprises applying a mask over the plurality of binding areas, depositing (e.g., using atomic layer deposition) a metal layer over the mask, and lifting the mask. Additional insulating material (e.g., an oxide (such as silicon dioxide, etc.) or nitride between approximately 3 nm and approximately 20 nm thick) may then be deposited over the plurality of additional lines and the plurality of binding areas after lifting the mask.

Also disclosed herein are methods of sequencing nucleic acid using the disclosed nucleic acid sequencing apparatuses. In some embodiments, a method comprises (a) binding at least one nucleic acid strand to the first binding area, (b) in one or more rounds of addition, adding, to the first binding area, an extendable primer and nucleic acid polymerase, (c) adding, to the first binding area, a first nucleotide precursor, the first nucleotide precursor labeled by a first cleavable magnetic label, and (d) sequencing the nucleic acid strand. The first cleavable magnetic label may comprise a magnetic nanoparticle (e.g., a molecule, a superparamagnetic nanoparticle, a ferromagnetic nanoparticle, etc.). The first binding area may be washed before step (c). Additional molecules of the nucleic acid polymerase may be added to the first binding area after step (c). Steps (c) and (d) may be repeated with a different nucleotide precursor during each repetition, each of the different nucleotide precursors being magnetically labeled. The first nucleotide precursor may comprise one of dATP, dGTP, dCTP, dTTP, or equivalents. Each of the first and different nucleotide precursors may be selected from magnetically-labeled adenine, guanine, cytosine, thymine, or their equivalents.

Sequencing the nucleic acid strand may comprise using the at least one line to detect the characteristic of the first magnetic sensor, the characteristic indicating presence or absence of the first cleavable magnetic label. The characteristic may be, for example, a magnetic field or a resistance, a frequency of a signal associated with or generated by a magnetic oscillator, a noise level, or a change in magnetic field or a change in resistance. The characteristic may result from a change in magnetic field or a change in resistance.

The method may also include a step of amplifying the at least one nucleic acid strand. If done, the amplification step may be done before or after binding the at least one nucleic acid strand to the first binding area. As a result of the amplifying, one or more amplicons may be bound to the first binding area.

In some embodiments, in response to a determination that the characteristic indicates the presence of the one or more magnetic nanoparticles coupled to the first binding area, a complementary base of the first nucleotide precursor is recorded in a record of a nucleic acid sequence of the nucleic acid strand.

In some embodiments, the first nucleotide precursor is nonextendable by the nucleic acid polymerase, and the method further comprises after detecting the characteristic, removing the first cleavable magnetic label and rendering the first nucleotide precursor extendable by the nucleic acid polymerase. In some embodiments, the first nucleotide precursor is not extendable by the nucleic acid polymerase. The first nucleotide precursor may be rendered extendable by chemical cleavage.

After sequencing the nucleic acid strand, the cleavable magnetic label may be removed by enzymatic or chemical cleavage.

In some embodiments, the first cleavable magnetic label has a first magnetic property, and the method further comprises, in the one or more rounds of addition, adding, to the first binding area, a second nucleotide precursor labeled by a second cleavable magnetic label having a second magnetic property. In some such embodiments, the method further comprises, in the one or more rounds of addition, adding, to the first binding area, a third nucleotide precursor labeled by a third cleavable magnetic label having a third magnetic property, and a fourth nucleotide precursor labeled by a fourth cleavable magnetic label having a fourth magnetic property.

In some embodiments, binding the at least one nucleic acid strand to the first binding area comprises attaching an adapter to an end of a respective one of the at least one nucleic acid strand, and coupling an oligonucleotide to the first binding area, wherein the oligonucleotide is capable of hybridizing to the adapter. In some embodiments, binding the at least one nucleic acid strand to the first binding area comprises covalently bonding each of the at least one nucleic acid strand to the first binding area. In some embodiments, binding the at least one nucleic acid strand to the first binding area comprises immobilizing the at least one nucleic acid strand via irreversible passive adsorption or affinity between molecules. In some embodiments, the first binding area comprises a cavity or a ridge, and binding the at least one nucleic acid strand to the first binding area comprises applying a hydrogel to the cavity or to the ridge.

In some embodiments, the nucleic acid polymerase is a Type B polymerase lacking 3′-5′ exonuclease activity. In some embodiments, the nucleic acid polymerase is a thermostable polymerase.

In some embodiments, using the at least one line comprises applying a current to the at least one line.

### Magnetic Labels

Methods for nucleic acid sequencing described herein rely on the use of magnetically-labeled nucleotide precursors comprising cleavable magnetic labels. These cleavable magnetic labels may comprise, for example, a magnetic nanoparticle, such as, for example, a molecule, a superparamagnetic nanoparticle, or a ferromagnetic particle. The magnetic labels may be nanoparticles with high magnetic anisotropy. Examples of nanoparticles with high magnetic anisotropy include, but are not limited to, Fe₃O₄, FePt, FePd, and CoPt. To facilitate chemical binding to nucleotides, the particles may be synthesized and coated with SiO₂. See, e.g., M. Aslam, L. Fu, S. Li, and V. P. Dravid, “Silica encapsulation and magnetic properties of FePt nanoparticles,” Journal of Colloid and Interface Science, Volume 290, Issue 2, 15 Oct. 2005, pp. 444-449. Because magnetic labels of this size have permanent magnetic moments, the directions of which fluctuate randomly on very short time scales, some embodiments, described further below, rely on sensitive sensing schemes that detect fluctuations in magnetic field caused by the presence of the magnetic labels.

There are a number of ways to attach the magnetic labels to nucleotide precursors and to cleave the magnetic labels after incorporation of the nucleotide precursor. For example, the magnetic labels may be attached to a base, in which case they may be cleaved chemically. As another example, the magnetic labels may be attached to a phosphate, in which case they may be cleaved by polymerase or, if attached via a linker, by cleaving the linker.

In some embodiments, the magnetic label is linked to the nitrogenous base (A, C, T, G, or a derivative) of the nucleotide precursor. After incorporation of the nucleotide precursor and the detection by a sequencing device (e.g., as described in further detail below), the magnetic label is cleaved from the incorporated nucleotide.

In some embodiments, the magnetic label is attached via a cleavable linker. Cleavable linkers are known in the art and have been described, e.g., in U.S. Pat. Nos. 7,057,026, 7,414,116 and continuations and improvements thereof. In some embodiments, the magnetic label is attached to the 5-position in pyrimidines or the 7-position in purines via a linker comprising an allyl or azido group. In other embodiments, the linker comprises a disulfide, indole or a Sieber group. The linker may further contain one or more substituents selected from alkyl (C₁₋₆) or alkoxy (C₁₋₆), nitro, cyano, fluoro groups or groups with similar properties. Briefly, the linker can be cleaved by water-soluble phosphines or phosphine-based transition metal-containing catalysts. Other linkers and linker cleavage mechanisms are known in the art. For example, linkers comprising trityl, p-alkoxybenzyl esters and p-alkoxybenzyl amides and tert-butyloxycarbonyl (Boc) groups and the acetal system can be cleaved under acidic conditions by a proton-releasing cleavage agent. A thioacetal or other sulfur-containing linker can be cleaved using a thiophilic metals, such as nickel, silver or mercury. The cleavage protecting groups can also be considered for the preparation of suitable linker molecules. Ester- and disulfide containing linkers can be cleaved under reductive conditions. Linkers containing triisopropyl silane (TIPS) or t-butyldimethyl silane (TBDMS) can be cleaved in the presence of F ions. Photocleavable linkers cleaved by a wavelength that does not affect other components of the reaction mixture include linkers comprising O-nitrobenzyl groups. Linkers comprising benzyloxycarbonyl groups can be cleaved by Pd-based catalysts.

In some embodiments, the nucleotide precursor comprises a label attached to a polyphosphate moiety as described in, e.g., U.S. Pat. Nos. 7,405,281 and 8,058,031. Briefly, the nucleotide precursor comprises a nucleoside moiety and a chain of 3 or more phosphate groups where one or more of the oxygen atoms are optionally substituted, e.g., with S. The label may be attached to the α, β, γ or higher phosphate group (if present) directly or via a linker. In some embodiments, the label is attached to a phosphate group via a non-covalent linker as described, e.g., in U.S. Pat. No. 8,252,910. In some embodiments, the linker is a hydrocarbon selected from substituted or unsubstituted alkyl, substituted or unsubstituted heteroalkyl, substituted or unsubstituted aryl, substituted or unsubstituted heteroaryl, substituted or unsubstituted cycloalkyl, and substituted or unsubstituted heterocycloalkyl; see, e.g., U.S. Pat. No. 8,367,813. The linker may also comprise a nucleic acid strand; see, e.g., U.S. Pat. No. 9,464,107.

In embodiments in which the magnetic label is linked to a phosphate group, the nucleotide precursor is incorporated into the nascent chain by the nucleic acid polymerase, which also cleaves and releases the detectable magnetic label. In some embodiments, the magnetic label is removed by cleaving the linker, e.g., as described in U.S. Pat. No. 9,587,275.

In some embodiments, the nucleotide precursors are non-extendable “terminator” nucleotides, i.e., the nucleotides that have a 3′-end blocked from addition of the next nucleotide by a blocking “terminator” group. The blocking groups are reversible terminators that can be removed in order to continue the strand synthesis process as described herein. Attaching removable blocking groups to nucleotide precursors is known in the art. See, e.g., U.S. Pat. Nos. 7,541,444, 8,071,739 and continuations and improvements thereof. Briefly, the blocking group may comprise an allyl group that can be cleaved by reacting in aqueous solution with a metal-allyl complex in the presence of phosphine or nitrogen-phosphine ligands. Other examples of reversible terminator nucleotides used in sequencing by synthesis include the modified nucleotides described in International Application No. PCT/US2019/066670, filed Dec. 16, 2019 and entitled “3′-protected Nucleotides,” published on Jun. 25, 2020 as WO/2020/131759, which is hereby incorporated by reference in its entirety for all purposes.

### Magnetic Sensors

Embodiments disclosed herein use magnetic sensors to detect the presence of magnetic labels coupled to nucleotide precursors as, for example, described above.

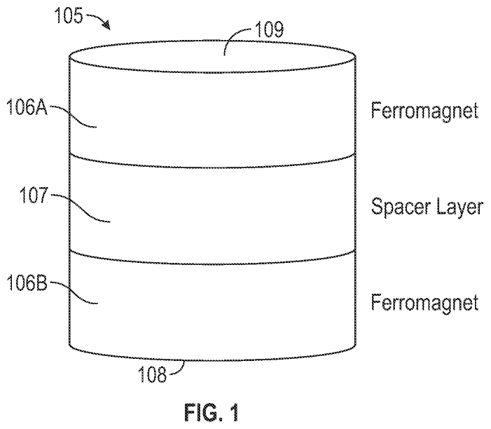

FIG. 1 illustrates a portion of a magnetic sensor 105 in accordance with some embodiments. The exemplary magnetic sensor 105 of FIG. 1 has a bottom surface 108 and a top surface 109 and comprises three layers, e.g., two ferromagnetic layers 106A, 106B separated by a nonmagnetic spacer layer 107. The nonmagnetic spacer layer 107 may be, for example, a metallic material such as, for example, copper or silver, in which case the structure is called a spin valve (SV), or it may be an insulator such as, for example, alumina or magnesium oxide, in which case the structure is referred to as a magnetic tunnel junction (MTJ). Suitable materials for use in the ferromagnetic layers 106A, 106B include, for example, alloys of Co, Ni, and Fe (sometimes mixed with other elements). In some embodiments, the ferromagnetic layers 106A, 106B are engineered to have their magnetic moments oriented either in the plane of the film or perpendicular to the plane of the film. Additional materials may be deposited both below and above the three layers 106A, 106B, and 107 shown in FIG. 1 to serve purposes such as interface smoothing, texturing, and protection from processing used to pattern the apparatus 100, but the active region of the magnetic sensor 105 lies in this trilayer structure. Thus, a component that is in contact with a magnetic sensor 105 may be in contact with one of the three layers 106A, 106B, or 107, or it may be in contact with another part of the magnetic sensor 105.

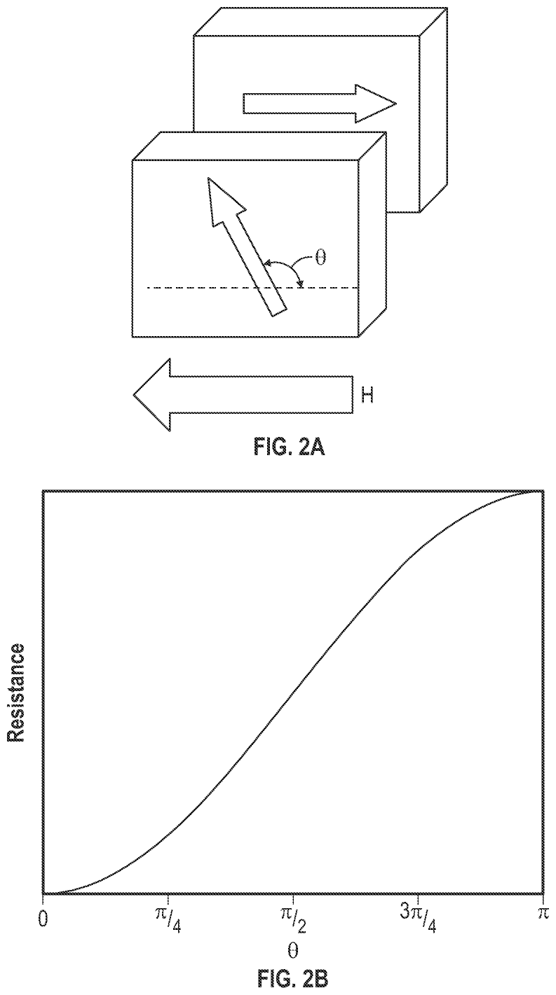

As shown in FIGS. 2A and 2B, the resistance of MR sensors is proportional to 1−cos(θ), where θ is the angle between the moments of the two ferromagnetic layers 106A, 106B shown in FIG. 1. To maximize the signal generated by a magnetic field and provide a linear response of the magnetic sensor 105 to an applied magnetic field, the magnetic sensor 105 may be designed such that the moments of the two ferromagnetic layers 106A, 106B are oriented π/2 or 90 degrees with respect to one another in the absence of a magnetic field. This orientation can be achieved by any number of methods that are known in the art. For example, one solution is to use an antiferromagnet to “pin” the magnetization direction of one of the ferromagnetic layers (either 106A or 106B, designated as “FM1”) through an effect called exchange biasing and then coat the sensor with a bilayer that has an insulating layer and permanent magnet. The insulating layer avoids electrical shorting of the magnetic sensor 105, and the permanent magnet supplies a “hard bias” magnetic field perpendicular to the pinned direction of FM1 that will then rotate the second ferromagnet (either 106B or 106A, designated as “FM2”) and produce the desired configuration. Magnetic fields parallel to FM1 then rotate FM2 about this 90 degree configuration, and the change in resistance results in a voltage signal that can be calibrated to measure the field acting upon the magnetic sensor 105. In this manner, the magnetic sensor 105 acts as a magnetic-field-to-voltage transducer.

Note that although the example discussed immediately above described the use of ferromagnets that have their moments oriented in the plane of the film at 90 degrees with respect to one another, a perpendicular configuration can alternatively be achieved by orienting the moment of one of the ferromagnetic layers 106A, 106B out of the plane of the film, which may be accomplished using what is referred to as perpendicular magnetic anisotropy (PMA).

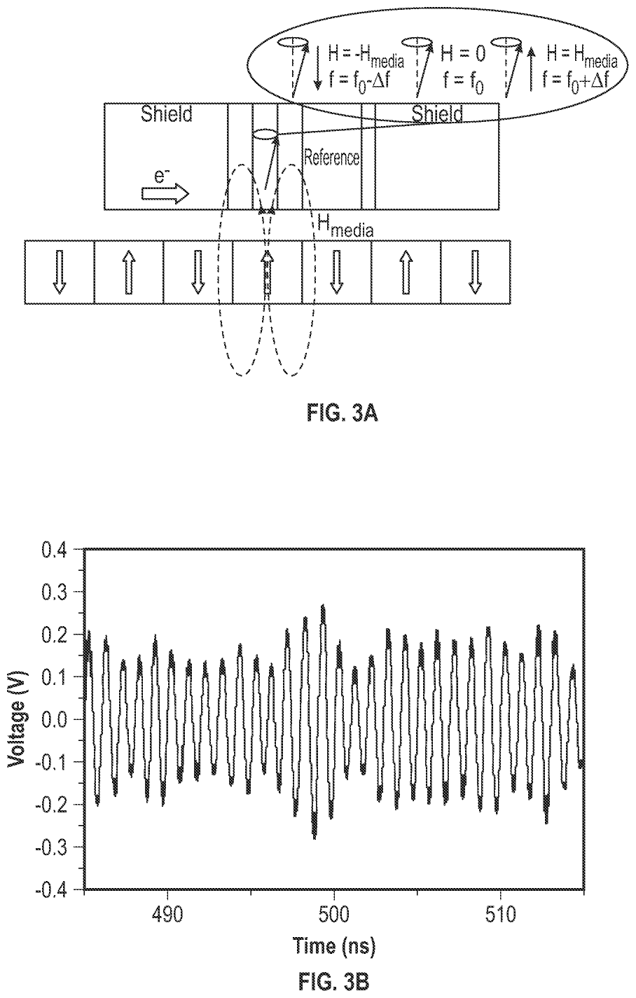

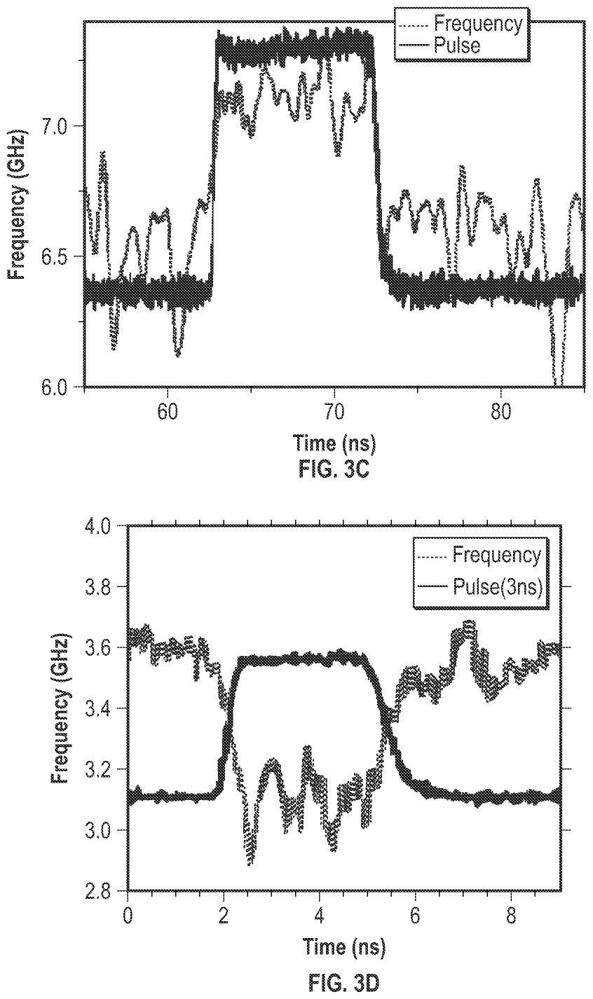

In some embodiments, the magnetic sensors 105 use a quantum mechanical effect known as spin transfer torque. In such devices, the electrical current passing through one ferromagnetic layer 106A (or 106B) in a SV or a MTJ preferentially allows electrons with spin parallel to the layer's moment to transmit through, while electrons with spin antiparallel are more likely to be reflected. In this manner, the electrical current becomes spin polarized, with more electrons of one spin type than the other. This spin-polarized current then interacts with the second ferromagnetic layer 106B (or 106A), exerting a torque on the layer's moment. This torque can in different circumstances either cause the moment of the second ferromagnetic layer 106B (or 106A) to precess around the effective magnetic field acting upon the ferromagnet, or it can cause the moment to reversibly switch between two orientations defined by a uniaxial anisotropy induced in the system. The resulting spin torque oscillators (STOs) are frequency-tunable by changing the magnetic field acting upon them. Thus, they have the capability to act as magnetic-field-to-frequency (or phase) transducers, as is shown in FIG. 3A, which illustrates the concept of using a STO sensor. FIG. 3B shows the experimental response of a STO through a delay detection circuit when an AC magnetic field with a frequency of 1 GHz and a peak-to-peak amplitude of 5 mT is applied across the STO. This result and those shown in FIGS. 3C and 3D for short nanosecond field pulses illustrate how these oscillators may be used as nanoscale magnetic field detectors. Further details may be found in T. Nagasawa, H. Suto, K. Kudo, T. Yang, K. Mizushima, and R. Sato, “Delay detection of frequency modulation signal from a spin-torque oscillator under a nanosecond-pulsed magnetic field,” Journal of Applied Physics, Vol. 111, 07C908 (2012).

### Apparatuses for Nucleic Acid Sequencing

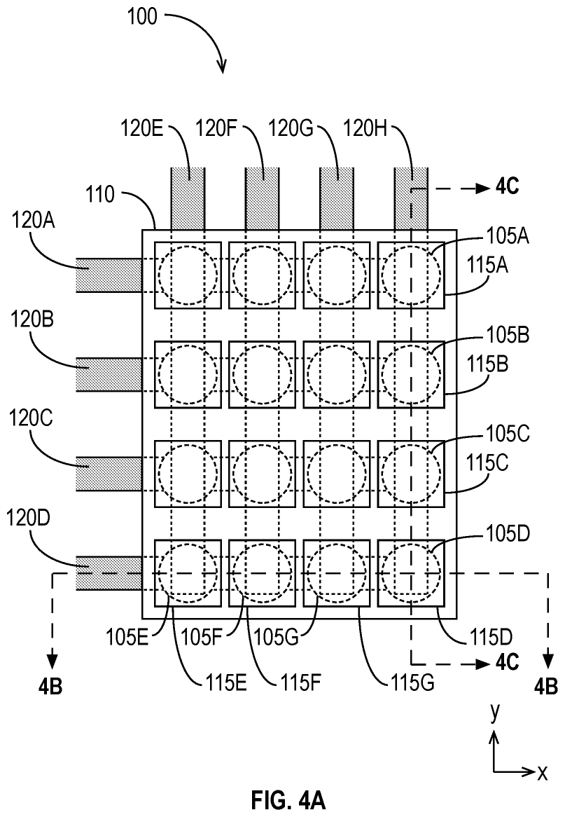

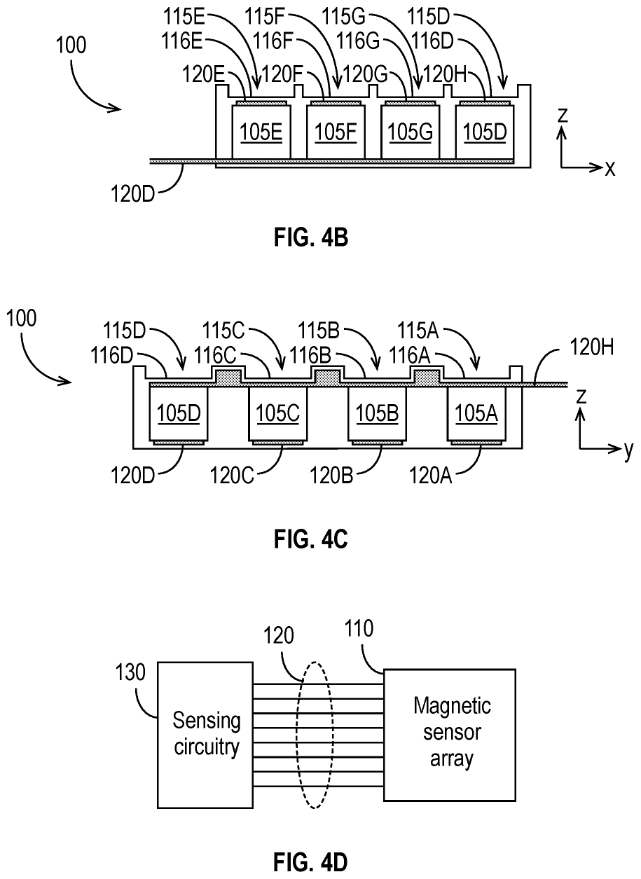

FIGS. 4A, 4B, and 4C illustrate portions of an apparatus 100 for nucleic acid sequencing in accordance with some embodiments. FIG. 4A is a top view of the apparatus. FIG. 4B is a cross-section view at the position indicated by the long-dash line labeled “4B” in FIG. 4A, and FIG. 4C is a cross-section view at the position indicated by the long-dash line labeled “4C” in FIG. 4A. As shown in FIG. 4A, the apparatus 100 comprises a magnetic sensor array 110 that includes a plurality of magnetic sensors 105, with sixteen magnetic sensors 105 shown in the array 110. To avoid obscuring the drawing, only seven of the magnetic sensors 105 are labeled in FIG. 4A, namely the magnetic sensors 105A, 105B, 105C, 105D, 105E, 105F, and 105G. (For simplicity, this document refers generally to the magnetic sensors by the reference number 105. Individual magnetic sensors are given the reference number 105 followed by a letter.) The apparatus 100 also includes at least one line 120, and, for at least some of the magnetic sensors 105, a binding area 115 for each of those magnetic sensors 105, both discussed in further detail below.

The magnetic sensors 105 and portions of the lines 120 within the magnetic sensor array 110 are illustrated using dashed lines to indicate that they might not be visible in the top view of the apparatus 100. As explained in further detail below, the magnetic sensors 105 are embedded in the apparatus 100 and are protected from the contents of the binding areas 115 (e.g., by an insulator). Accordingly, it is to be understood that the various illustrated components (e.g., lines 120, magnetic sensors 105, etc.) might not be visible in a physical instantiation of the apparatus 100 (e.g., they may be embedded in or covered by protective material, such as an insulator).

In some embodiments, each of the magnetic sensors 105 of the magnetic sensor array 110 is a thin film device that uses the magnetoresistance (MR) effect to detect magnetic labels in an associated binding area 115, described in further detail below. As described in more detail below, each magnetic sensor 105 may operate as a potentiometer with a resistance that varies as the strength and/or direction of the sensed magnetic field changes.

The exemplary magnetic sensor array 110 in the exemplary embodiment of FIG. 4A is a rectangular array, with the magnetic sensors 105 arranged in rows and columns. In other words, the plurality of magnetic sensors 105 of the magnetic sensor array 110 is arranged in a rectangular grid pattern. It is to be understood that the arrangement of magnetic sensors 105 in a grid pattern as shown in FIG. 4A is one of many possible arrangements. It will be appreciated by those having ordinary skill in the art that other arrangements of the magnetic sensors 105 are possible and are within the scope of the disclosures herein.

Referring now to FIGS. 4B and 4C in conjunction with FIG. 4A, each magnetic sensor 105 illustrated in the exemplary embodiment of the apparatus 100 has a cylindrical shape. It is to be understood, however, that in general the magnetic sensors 105 can have any suitable shape. For example, the magnetic sensors 105 may be cuboid in three dimensions. Moreover, different magnetic sensors 105 can have different shapes (e.g., some may be cuboid and others cylindrical, etc.).

As shown in the exemplary embodiment of FIGS. 4A, 4B, and 4C, a binding area 115 is disposed above each magnetic sensor 105. For example, the binding area 115A is above the magnetic sensor 105A; the binding area 115B is above the magnetic sensor 105B; the binding area 115C is above the magnetic sensor 105C; the binding area 115D is above the magnetic sensor 105D; the binding area 115E is above the magnetic sensor 105E; the binding area 115F is above the magnetic sensor 105F; and the binding area 115G is above the magnetic sensor 105G. Each of the other, unlabeled nine magnetic sensors 105 shown in FIG. 4A is also disposed below a corresponding binding area 115 (also unlabeled in FIG. 4A).

The binding areas 115 hold fluids. The magnetic sensors 105 are able to detect magnetic labels (e.g., nanoparticles) that are in the binding areas 115. Thus, in some embodiments the surface 116 of each binding area 115 has properties and characteristics that protect the magnetic sensors 105 from whatever fluids are in the binding areas 115, while still allowing the magnetic sensors 105 to detect magnetic labels that are within the binding areas 115. The material of the surface 116 (and possibly of the rest of the binding area 115) may be or comprise an insulator. For example, in some embodiments, the surface 116 comprises polypropylene, gold, glass, or silicon. It is to be understood that the surface 116 may be the exposed surface of a multi-layer structure disposed over the line(s) 120 that reside over the magnetic sensors 105. For example, in embodiments in which the surface 116 comprises a conductor (e.g., gold), a layer of an insulating material can be used to separate the conductor from the line(s) 120 over the magnetic sensors 105. (See FIGS. 4B and 4C.) The thickness of the surface 116 may be selected so that the magnetic sensors 105 are at a distance from the binding areas 115 such that the magnetic sensors 105 can detect magnetic labels within the binding areas 115. In some embodiments, the surface 116 is approximately 3 to 20 nm thick so that the sensing layer of a magnetic sensor 105 (described further below) is between approximately 5 nm and approximately 40 nm from the magnetic labels in its corresponding binding area 115.

In some embodiments, the surface 116 of a binding area 115 has a structure (or multiple structures) configured to anchor nucleic acid to the surface 116. For example, the structure (or structures) may include a cavity or a ridge. Furthermore, in some embodiments, the surface 116 has characteristics that promote amplification of nucleic acids. For example, the apparatus 100 may facilitate bridge amplification to promote the generation of clonal clusters of a single nucleic acid strand within each of the binding areas 115.

Each binding area 115 shown in the exemplary embodiment of FIGS. 4A, 4B, and 4C is cuboid in shape (e.g., as shown in FIG. 4A, each binding area 115 has a square shape when viewed from the top and is rectangular when viewed in cross-section), but it is to be appreciated that the binding areas 115 can have other shapes (e.g., circular, oval, octagonal, etc.). For example, the shapes of the binding areas can be similar or identical to the shapes of the magnetic sensors 105 (e.g., if the magnetic sensors 105 are cylindrical in three dimensions, the binding areas 115 can also be cylindrical with a radius that can be larger, smaller, or the same size as the radii of the magnetic sensors 105; if the magnetic sensors 105 are cuboid in three dimensions, the binding areas 115 can also be cuboid with a surface 116 that is larger, smaller, or the same size as the tops of the magnetic sensors 105, etc.). Moreover, different binding areas 115 and different surfaces 116 can have different shapes (e.g., some surfaces 116 can be circular, some can be rectangular, some can be square, etc.). Additionally, although FIGS. 4B and 4C show that the binding areas 115 have vertical sides, there is no requirement for the sides to be vertical. In general, the binding areas 115 and their surfaces 116 can have any shapes and characteristics that facilitate the detection of magnetic nanoparticles in the binding areas 115 by the magnetic sensors 105.

In some embodiments, such as the exemplary embodiment illustrated in FIGS. 4A, 4B, and 4C, each of the plurality of magnetic sensors 105 is coupled to at least one line 120. (For simplicity, this document refers generally to the lines by the reference number 120. Individual lines are given the reference number 120 followed by a letter.) In the exemplary embodiment shown in FIGS. 4A, 4B, and 4C, each magnetic sensor 105 of the magnetic sensor array 110 is coupled to two lines 120. For example, the magnetic sensor 105A is coupled to the lines 120A and 120H; the magnetic sensor 105B is coupled to the lines 120B and 120H; the magnetic sensor 105C is coupled to the lines 120C and 120H; the magnetic sensor 105D is coupled to the lines 120D and 120H; the magnetic sensor 105E is coupled to the lines 120D and 120E; the magnetic sensor 105F is coupled to the lines 120D and 120F; and the magnetic sensor 105G is coupled to the lines 120D and 120G. In the exemplary embodiment of FIGS. 4A, 4B, and 4C, the lines 120A, 120B, 120C, and 120D are shown residing under the magnetic sensors 105, and the lines 120E, 120F, 120G, and 120H are shown residing above the magnetic sensors 105.

FIG. 4B shows the magnetic sensor 105E in relation to the lines 120D and 120E, the magnetic sensor 105F in relation to the lines 120D and 120F, the magnetic sensor 105G in relation to the lines 120D and 120G, and the magnetic sensor 105D in relation to the lines 120D and 120H. FIG. 4C shows the magnetic sensor 105D in relation to the lines 120D and 120H, the magnetic sensor 105C in relation to the lines 120C and 120H, the magnetic sensor 105B in relation to the lines 120B and 120H, and the magnetic sensor 105A in relation to the lines 120A and 120H.

Each of the lines 120 in the exemplary embodiment of FIGS. 4A, 4B, and 4C identifies a row or a column of the magnetic sensor array 110. For example, each of the lines 120A, 120B, 120C, and 120D identifies a different row of the magnetic sensor array 110, and each of the lines 120E, 120F, 120G, and 120H identifies a different column of the magnetic sensor array 110. As shown in FIG. 4B, each of the lines 120E, 120F, 120G, and 120H is in contact with one of the magnetics sensors 105 along the cross-section (namely, line 120E is in contact with the top of magnetic sensor 105E, line 120F is in contact with the top of magnetic sensor 105F, line 120G is in contact with the top of magnetic sensor 105G, and line 120H is in contact with the top of magnetic sensor 105D), and the line 120D is in contact with the bottom of each of the magnetic sensors 105E, 105F, 105G, and 105D. Similarly, and as shown in FIG. 4C, each of the lines 120A, 120B, 120C, and 120D is in contact with the bottom of one of the magnetic sensors 105 along the cross-section (namely, line 120A is in contact with the bottom of magnetic sensor 105A, line 120B is in contact with the bottom of magnetic sensor 105B, line 120C is in contact with the bottom of magnetic sensor 105C, and line 120D is in contact with the bottom of magnetic sensor 105D), and the line 120H is in contact with the top of each of the magnetic sensors 105D, 105C, 105B, and 105A.

In some embodiments, some or all of the binding areas 115 reside in trenches in the lines 120 passing over the magnetic sensors 105. For example, as shown in FIG. 4C, the line 120H is thinner over the magnetic sensors 105 than it is between the magnetic sensors 105. For example the line 120H has a first thickness above the magnetic sensor 105D, a second, larger thickness between the magnetic sensors 105D and 105C, and the first thickness above the magnetic sensor 105C.

To simplify the explanation, FIGS. 4A, 4B, and 4C illustrate an exemplary apparatus 100 with only sixteen magnetic sensors 105 in the magnetic sensor array 110, only sixteen corresponding binding areas 115, and eight lines 120. It is to be appreciated that the apparatus 100 may have fewer or many more magnetic sensors 105 in the magnetic sensor array 110, and it may have more or fewer binding areas 115, and it may have more or fewer lines 120. In general, any configuration of magnetic sensors 105 and binding areas 115 that allows the magnetic sensors 105 to detect magnetic labels in the binding areas 115 may be used. Similarly, any configuration of one or more lines 120 that allows the determination of whether the magnetic sensors 105 have sensed one or more magnetic labels may be used.

FIG. 4D is a block diagram showing components of the apparatus 100 in accordance with some embodiments. As illustrated, the apparatus 100 includes the magnetic sensor array 110, which is coupled to sensing circuitry 130 by the lines 120. In operation, the sensing circuitry 130 can apply a current to the lines 120 to detect a characteristic of at least one of the plurality of magnetic sensors 105 in the magnetic sensor array 110, where the characteristic indicates a presence or an absence of a magnetically-labeled nucleotide precursor in the binding area 115. For example, in some embodiments, the characteristic is a magnetic field or a resistance, or a change in magnetic field or a change in resistance. In some embodiments, the characteristic is a noise level. In some embodiments, the magnetic sensor comprises a magnetic oscillator, and the characteristic is a frequency of a signal associated with or generated by the magnetic oscillator.

In some embodiments, the sensing circuitry 130 detects deviations or fluctuations in the magnetic environment of some or all of the magnetic sensors 105 in the magnetic sensor array 110. For example, a magnetic sensor 105 of the MR type in the absence of a magnetic label should have relatively small noise above a certain frequency as compared to a magnetic sensor 105 in the presence of a magnetic label, because the field fluctuations from the magnetic label will cause fluctuations of the moment of the sensing ferromagnet. These fluctuations can be measured using heterodyne detection (e.g., by measuring noise power density) or by directly measuring the voltage of the magnetic sensor 105 and evaluated using a comparator circuit to compare to a dummy sensor element that does not sense the binding area 115. In the case the magnetic sensors 105 include STO elements, fluctuating magnetic fields from magnetic labels would cause jumps in phase for the magnetic sensors due to instantaneous changes in frequency, which can be detected using a phase detection circuit. Another option is to design the STO such that it oscillates only within a small magnetic field range such that the presence of a magnetic label would turn off the oscillations. It is to be understood that the examples provided above are merely exemplary. Other detection methodologies are contemplated and are within the scope of this disclosure.

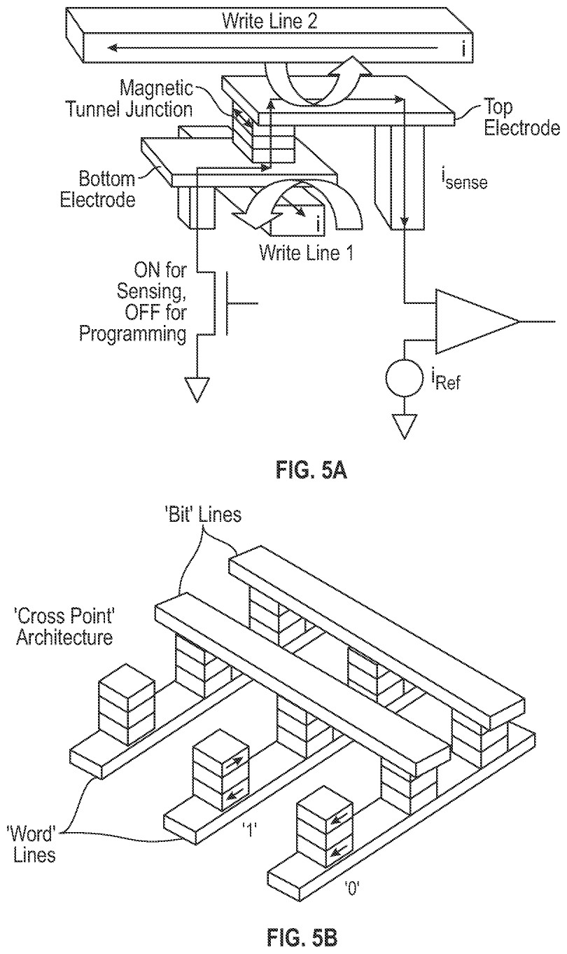

In some embodiments, the magnetic sensor array 110 includes a selector element that reduces the chances of “sneak” currents that could transmit through neighboring elements and degrade the performance of the magnetic sensor array 110. FIGS. 5A and 5B illustrate two approaches in accordance with some embodiments. In FIG. 5A, a CMOS transistor is coupled in series with the magnetic sensor 105. For more detail on the configuration shown in FIG. 5A, see B. N. Engel, J. Akerman, B. Butcher, R. W. Dave, M. DeHerrera, M. Durlam, G. Grynkewich, J. Janesky, S. V. Pietambaram, N. D. Rizzo, J. M. Slaughter, K. Smith, J. J. Sun, and S. Tehrani, “A 4-Mb Toggle MRAM Based on a Novel Bit and Switching Method,” IEEE Transactions on Magnetics, Vol. 41, 132 (2005).

In FIG. 5B, a diode or a diode-like element is deposited together with the magnetic films and then placed into a “cross-point” architecture, where CMOS transistors at the periphery of the magnetic sensor array 110 turn on the individual lines 120 (e.g., word and bit lines) to address individual magnetic sensors 105 in the array. The use of CMOS select transistors may be simpler due to the prevalence of foundries available to fabricate the front end (e.g., all the nanofabrication to build the CMOS transistors and underlying circuitry), but the types of currents required for operation may require a cross-point design to eventually reach the densities required of the magnetic sensor array 110. Additional details on the configuration shown in FIG. 5B may be found in C. Chappert, A. Fert, and F. N. Van Daul, “The emergence of spin electronics in data storage,” Nature Materials, Vol. 6, 813 (2007).

In some embodiments, use of the apparatus 100 allows for amplification of nucleic acid, such as, for example, using bridge amplification (discussed further below). The distances between the individual strands in the clonal clusters created through an amplification procedure, such as described below in more detail, can be estimated to select the size(s) and density of magnetic sensors 105 in the magnetic sensor array 110. For example, to estimate the distances, one can consider both the contour length (e.g., the length of a straightened-out strand of DNA) and the persistence length (e.g., the average length of a strand after it bends over during a bridge amplification procedure) of a 200 base pair (BP) double-stranded DNA, chosen because the average strand length for many nucleic acid sequencing applications is 200 BP, and double-stranded DNA is less flexible than single-stranded DNA and therefore provides an upper bound. The average contour length is about 65 nm, and the persistence length is approximately 35 nm (see, e.g., S. Brinkers et al., “The persistence length of double stranded DNA determined using dark field tethered particle motion,” J. Chem. Phys. (2009) 130:215105). Because the DNA bends over during the bridge amplification process, the average distance between amplified clones should be somewhere between the contour length and the persistence length. Accordingly, that distance can be estimated to be approximately 40 nm. Assuming that, from a signal-to-noise ratio (SNR) perspective, it may be desirable to have between tens and hundreds of copied strands to sequence each originally-attached target strand, the magnetic sensors 105 can have dimensions on the order of, for example, approximately 10 nm to approximately 1 μm. It is to be appreciated that because the sequencing uses magnetic nanoparticles and not fluorescence, the spacing between adjacent magnetic sensors 105 can be much smaller than that required for optical system, which are limited by diffraction effects. For example, in embodiments of the apparatus 100 disclosed herein, adjacent magnetic sensors 105 may be between approximately 20 nm and approximately 30 nm apart.

### Method(s) of Manufacturing a Sequencing Apparatus

In some embodiments, the apparatus 100 is fabricated using photolithographic processes and thin film deposition.

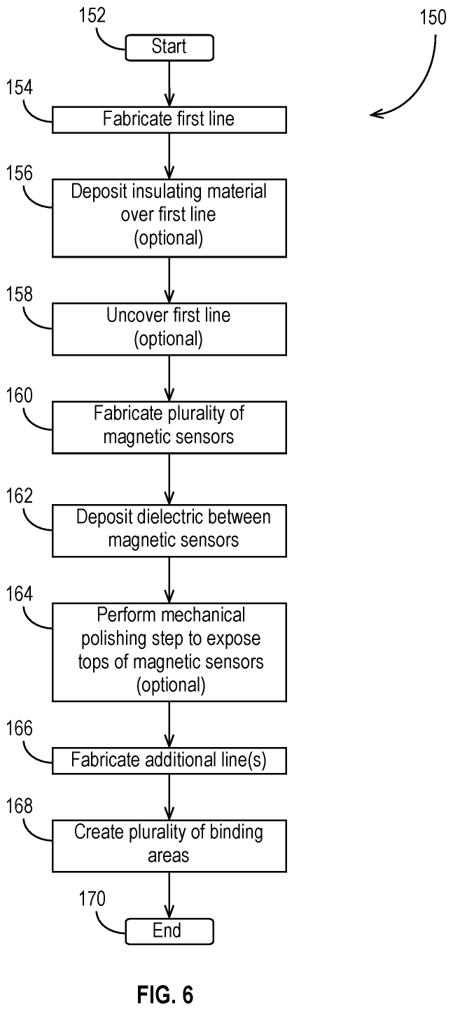

FIG. 6 illustrates a method 150 of manufacturing the apparatus 100 in accordance with some embodiments. At 152, the method begins. At 154, at least one line 120 (e.g., a first line 120) is fabricated on a substrate, for example, by depositing a metal layer on a substrate, and patterning the metal layer into the at least one line 120. The metal layer may be deposited, for example, using physical vapor deposition (PVD) or ion beam deposition (IBD). Patterning the metal layer in to the at least one line 120 can be accomplished using photolithography, milling, and/or etching.

Optionally, at 156, insulating material may be deposited over the at least one line 120, and then, also optionally, at 158, the at least one line 120 can be uncovered. For example, the at least one line 120 can be uncovered using chemical mechanical polishing (CMP).

At 160, a plurality of magnetic sensors 105 (e.g., the magnetic sensor array 110) is fabricated on the at least one line 120. The plurality of magnetic sensors 105 may be fabricated, for example, by depositing a plurality of layers on the at least one line 120, and then patterning the plurality of layers to form the plurality of magnetic sensors 105. The plurality of layers may be deposited using any suitable technique. For example, the plurality of layers may be deposited by depositing a first ferromagnetic layer (e.g., the layer 106B shown in FIG. 1), depositing a metal or insulator layer (e.g., the layer 107 shown in FIG. 1) over the first ferromagnetic layer, and depositing a second ferromagnetic layer (e.g., the layer 106A shown in FIG. 1) over the metal or insulator layer. Patterning the plurality of layers to form the plurality of magnetic sensors 105 can be accomplished using any suitable technique, such as, for example, photolithography or etching.

In some embodiments, each magnetic sensor 105 of the magnetic sensor array 110 has a bottom surface 108 and a top surface 109. (See, e.g., FIG. 1.) The bottom surface 108 is coupled to one of the at least one line 120 (e.g., the bottom surface 108 is coupled to the first line 120). In some embodiments, the bottom surface 108 of each magnetic sensor 105 is in contact with one of the at least one line 120 (e.g., the first line 120).

In some embodiments, each of the plurality of magnetic sensors 105 has a predetermined shape, which may be the same for all magnetic sensors 105 of the plurality of magnetic sensors 105 or different for two or more magnetic sensors 105. The predetermined shape may be any suitable shape, including, for example, substantially cylindrical or substantially cuboid. A lateral dimension of each of the plurality of magnetic sensors 105 may be, for example, between approximately 10 nm and approximately 1 μm. As used herein, the term “lateral dimension” means a dimension in the x-y plane shown in FIG. 4A, e.g., when the apparatus 100 is viewed from the top. For example, when a magnetic sensor 105 is cylindrical, a lateral dimension is the diameter of the top surface 109 of the cylinder. As another example, when a magnetic sensor 105 is cuboid, its lateral dimensions include the dimensions of its top surface (e.g., the length, width, or diagonal dimension(s) of its top surface 109).

Referring again to the method embodiment of FIG. 6, at 162, insulating material (e.g., dielectric material) is deposited between the magnetic sensors 105 of the magnetic sensor array 110. The insulating material may be any suitable material, such as, for example, an oxide or nitride. For example, the insulating material may comprise silicon dioxide (SiO₂), aluminum oxide (Al₂O₃), or silicon nitride (Si₃N₄).

Optionally, at 164, a chemical mechanical polishing step may be performed to expose the top surface 109 of each of the plurality of magnetic sensors 105.

At 166, at least one additional line 120 is fabricated using any suitable technique. For example, the at least one additional line 120 may be fabricated by depositing a layer of metal, performing photolithography to define the at least one additional line 120, and removing a portion of the layer of metal, thereby leaving the at least one additional line 120.

In some embodiments, each of the at least one additional line 120 is coupled to the top surface 109 of at least one magnetic sensor 105 in the magnetic sensor array 110. In some embodiments, the top surface 109 of each magnetic sensor 105 is in contact with the same line 120. In some embodiments, the bottom surface 108 of a magnetic sensor 105 is in contact with a first line 120A, and the top surface 109 of the magnetic sensor 105 is in contact with a second line 120B.

In some embodiments, the plurality of magnetic sensors 105 is in a rectangular magnetic sensor array 110. In such embodiments, the at least one line 120 (e.g., the first or bottom line 120) may correspond to one or more rows of the rectangular array, and the at least one additional line 120 (e.g., the second or top line 120) may correspond to one or more columns of the rectangular array, or vice versa.

At 168, a plurality of binding areas 115 is created using any suitable technique. For example, the plurality of binding areas 115 may be created by applying a mask over the regions corresponding to the plurality of binding areas 115, depositing a metal layer over the mask, and lifting the mask. For example, photolithography may be performed to define a mask with windows in polymer overlapping the top lines 120, except immediately above the magnetic sensors 105. A subsequent metal deposition and lift-off may then be performed to thicken the top lines 120 away from the magnetic sensors 105, which creates a shallow trench above each magnetic sensor 105 and reduces the resistance of the top lines 120 to improve noise performance. These shallow trenches can define the binding areas 115.

Accordingly, in some embodiments, the plurality of binding areas 115 is created by making a trench in a top line 120 at a position corresponding to the top of a magnetic sensor 105, and then depositing insulating material over the trench. For example, in embodiments in which the plurality of magnetic sensors 105 is arranged in a rectangular array 110, with some lines 120 (the bottom lines 120) below the magnetic sensors 105 and other lines 120 (the top lines 120) above the magnetic sensors 105, a trench may be etched in each of the top lines 120 at the locations where they pass over the magnetic sensors 105. The binding areas 115 are then defined by the trenches above the magnetic sensors 105 (e.g., as shown in FIG. 4C).

In some embodiments, after creating the binding areas 115 (e.g., after lifting the mask and/or creating the trenches described above), a thin layer of additional insulating material (e.g., an oxide such as SiO₂ or a nitride) is deposited (e.g., using atomic layer deposition) over the plurality of additional lines 120 and the plurality of binding areas 115. The thickness of the additional insulating material may be, for example, between approximately 3 nm and approximately 20 nm. Any suitable insulating material that electrically isolates the magnetic sensors 105 from magnetic labels in the binding areas 115 and protects the magnetic sensors 105 from fluids expected to be added to the binding areas 115 may be used for this purpose. For example, the additional insulating material may comprise silicon dioxide (SiO₂), aluminum oxide (AlOₓ), or silicon nitride (SiN), or the like.

At 170, the method 150 ends.

### Methods of Sequencing

In some embodiments, nucleic acid is sequenced using immobilized nucleic acid strands (potentially in clonal clusters) that are tethered to the apparatus 100 in the proximity of the magnetic sensors 105 of the magnetic sensor array 110. Four types of reversible terminator bases (RT-bases) may then be added, either together or one at a time, and non-incorporated nucleotides are washed away. Then the magnetic labels, along with the terminal 3′ blocker, may be chemically removed from the nucleic acid strands before the next sequencing cycle begins.

The nucleic acid strands can be prepared in any suitable manner. For example, the nucleic acid strands can be prepared by random fragmentation of a nucleic acid sample, followed by 5′and 3′ adapter ligation. These strands of the nucleic acid may then be captured on oligos bound or attached to the surfaces 116 of at least some of the binding areas 115. Linear or exponential amplification including bridge amplification may be used to amplify the strands prior to sequencing.

Bridge amplification and other amplification techniques are well known in the art and can be used with the apparatus 100 in accordance with some embodiments. To begin bridge amplification, the nucleic acid to be sequenced can be attached to a substrate using, for example, adapter strands that are, for example, immobilized in a hydrogel. Then polymerase, primers, and nucleotide precursors may be introduced into the binding area 115 to create double-stranded nucleic acids from the single target strands. Next, the double strands are denatured, which separates the double-sided nucleic acid strands into two single strands that are complements of each other. Bridge formation involves chemistry to cause the single strands to fold over and attach to the complementary adapter strands immobilized on the substrate as shown. Once again, polymerase, primers, and nucleotide precursors are introduced into the binding area 115 to convert individual single strand “bridges” into double-sided strands. Following this step, the double strands are denatured to produce complementary single strands, one being the original “forward” strand and the other a copied “reverse” strand. After repeating these steps many times, clonal clusters are formed with both forward and reverse copies. One of the two clusters (e.g., the reverse strands) can then be cleaved from the binding area 115 before sequencing the remaining cluster (e.g., the forward strands).

The use of an amplification procedure in connection with the apparatus 100 for nucleic acid sequencing can improve the SNR of the sequencing process and thereby improve accuracy of the sequencing. The SNR improvement results because the presence of many copies of the same nucleic acid strand to be sequenced within a binding area 115 allows a larger number of magnetically-labeled nucleotide precursors to be incorporated within the binding area 115. The incorporation of a larger number of magnetically-labeled nucleotide precursors, in turn, increases the likelihood that the magnetic sensor 105 associated with that binding area 115 will detect the presence of the magnetic labels within the binding area 115. Thus, having a larger number of copies of the strand to be sequenced reduces the likelihood that the magnetic sensor 105 will miss the incorporation of a magnetically-labeled nucleotide precursor and thereby make a sequencing error.

To sequence the nucleic acid strands, magnetically-labeled nucleotide precursors may be introduced one at a time or all at once, as described below.

In some embodiments, magnetically-labeled nucleotide precursors are introduced one at a time. In such embodiments, the same magnetic label can be used for all of the nucleotide precursors. It is to be understood that as used herein, the phrase “the same magnetic label” does not refer to the same physical instance of a single magnetic label (i.e., it does not mean that a particular instance of a physical label is reused); instead, it refers to multiple physical instantiations of magnetic labels, all of which have identical characteristics or properties that render individual instances of them indistinguishable from one another. In contrast, the phrase “different magnetic labels” refers to magnetic labels that, either individually or as a group, have different characteristics or properties that allow them to be distinguished from other magnetic labels, whether individually or as a group.

In some embodiments, the nucleic acid strands are extended one nucleotide at a time, and the magnetic sensor array 110 is used to identify the bound magnetically-labeled nucleotide precursors.

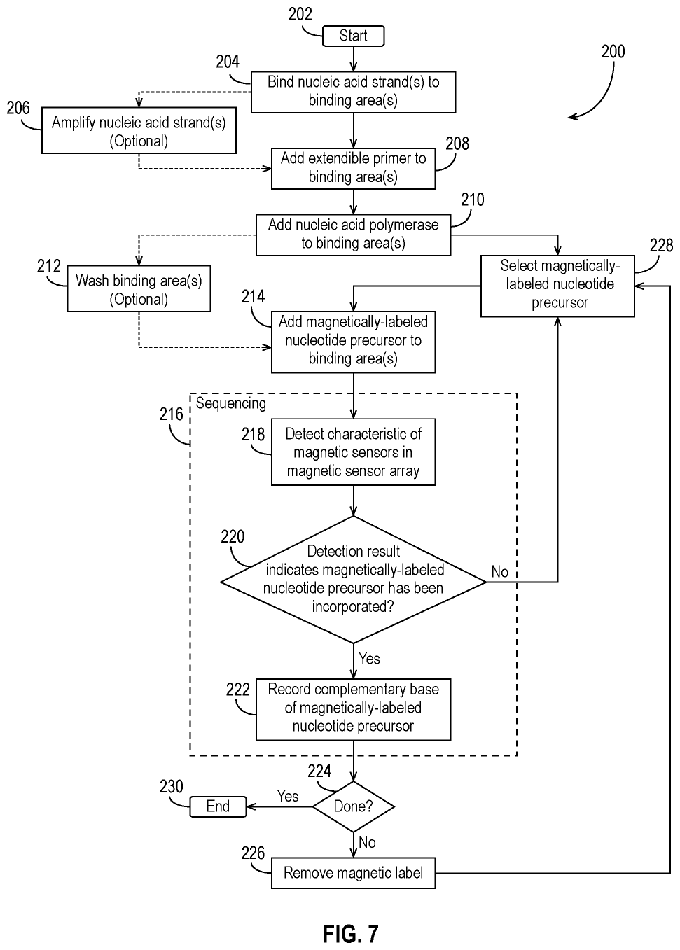

FIG. 7 is a flowchart illustrating a method 200 of using the apparatus 100, or another apparatus that senses the presence or absence of magnetic labels using magnetic sensors, for nucleic acid sequencing in accordance with some embodiments. At 202, the method begins. At 204, one or more nucleic acid strands are bound to the surface 116 of one or more binding areas 115 of the sequencing apparatus 100, as described above. There are a number of ways to bind the one or more nucleic acid strands to the surface 116. For example, the nucleic acid strand may be bound to the surface 116 by attaching an adapter to an end of the nucleic acid strand and coupling an oligonucleotide to the surface 116 of the binding area 115, wherein the oligonucleotide is complementary to the adapter. As another example, the nucleic acid strand may be bound to the surface 116 by covalently bonding the nucleic acid strand to the surface 116. As yet another example, the nucleic acid strand may be bound to the surface 116 by immobilizing the nucleic acid strand via irreversible passive adsorption or affinity between molecules. In some embodiments, the surface 116 comprises a cavity or a ridge, as described above, and binding the nucleic acid strand to the proximal wall comprises applying a hydrogel to the cavity or to the ridge.

At optional step 206, the nucleic acid strand(s) may be amplified using any suitable method, such as, for example, by leveraging the polymerase chain reaction (PCR) or linear amplification.

At 208, an extendible primer is added to the binding area 115.

At 210, a nucleic acid polymerase is added to the binding area 115. The nucleic acid polymerase may be any suitable nucleic acid polymerase. Desired characteristics of a nucleic acid polymerase (such as a DNA polymerase) that finds use in nucleic acid sequencing include one or more of the following: fast association rate for nucleic acid template and for nucleotide precursors or slow dissociation rate for nucleic acid template and for nucleotide precursors (association and dissociation rates being kinetic characteristics of a nucleic acid polymerase under a defined set of reaction conditions); high fidelity, low or undetectable exonuclease activity, including low or undetectable 3′-5′ exonuclease (proofreading) activity or low or undetectable 5′-3′ exonuclease activity; effective DNA strand displacement, high stability, high processivity (including long read length), salt tolerance and ability to incorporate modified nucleotide precursors including the precursors described herein.

Some examples of a suitable polymerase include B-family (Type B) polymerases lacking the 3′-5′ exonuclease activity.

In some embodiments, the polymerase is a thermostable polymerase. Thermostable nucleic acid polymerases include Thermus aquaticus Taq DNA polymerase, Thermus sp. Z05 polymerase, Thermus flavus polymerase, Thermotoga maritima polymerases, such as TMA-25 and TMA-30 polymerases, Tth DNA polymerase, Pyrococcus furiosus (Pfu), Pyrococcus woesei (Pwo), Thermatoga maritima (Tma) and Thermococcus Litoralis (Tli or Vent) and the like.

In some embodiments, the polymerase lacks detectable 5′-3′ exonuclease activity. Examples of DNA polymerases substantially lacking 5′ to 3′ nuclease activity include the Klenow fragment of E. coli DNA polymerase I; a Thermus aquaticus DNA polymerase (Taq) lacking the N-terminal 235 amino acids (“Stoffel fragment”), See U.S. Pat. No. 5,616,494. Other examples include a thermostable DNA polymerase having sufficient deletions (e.g., N-terminal deletions), mutations, or modifications so as to eliminate or inactivate the domain responsible for the 5′-3′ nuclease activity. See, e.g., U.S. Pat. No. 5,795,762.

In some embodiments, the polymerase lacks detectable 3′-5′ exonuclease activity. Examples of DNA polymerases substantially lacking the 3′-5′ exonuclease activity include the Taq polymerase and its derivatives and any B-family (Type B) polymerase with naturally occurring or engineered deletion of the proofreading domain.

In some embodiments, the polymerase has been modified or engineered to enable or enhance incorporation of nucleotide analogs such as 3′-modified nucleotides; see, e.g., U.S. Pat. Nos. 10,150,454, 9,677,057, and 9,273,352.

In some embodiments, the polymerase has been modified or engineered to enable or enhance incorporation of nucleotide analogs such as 5′-phosphate-modified nucleotides; see, e.g., U.S. Pat. Nos. 10,167,455 and 8,999,676. In some embodiments, such polymerases are phi29 derived polymerases; see, e.g., U.S. Pat. Nos. 8,257,954 and 8,420,366. In some embodiments, such polymerases are phiCPV4 derived polymerases; see, e.g., U.S. Patent Publication No. US20180245147.

In some embodiments, the polymerase is modified or engineered by selection to successfully incorporate a desired modified nucleotide or to incorporate nucleotides and nucleotide analogs with desired accuracy and processivity. Methods of selecting such modified polymerases are known in the art; see, e.g., U.S. Patent Publication No. US20180312904A1, entitled “Polymerase Compositions and Methods of Making and Using Same.”

It is to be understood that steps 208 and 210 may be combined or their order reversed.

Optionally, at 212, the binding area 115 may be washed before adding the magnetically-labeled nucleotide precursor at step 214.

At 228, a magnetically-labeled nucleotide precursor is selected for the sequencing cycle. In some embodiments, the magnetically-labeled nucleotide precursor is selected from adenine, guanine, cytosine, thymine, or their equivalents. In some embodiments, the magnetically-labeled nucleotide precursor comprises one of magnetically-labeled dATP, dGTP, dCTP, dTTP, or equivalents. The magnetically-labeled nucleotide precursor may be labeled conventional, natural, unconventional, or an analog nucleotide. The term “conventional” or “natural” when referring nucleotide precursors refers to those occurring naturally (i.e., for DNA these are dATP, dGTP, dCTP and dTTP). The term “unconventional” or “analog” when referring to nucleotide precursors includes modifications or analogues of conventional bases, sugar moieties, or inter-nucleotide linkages in nucleotide precursors. For example, dITP, 7-deaza-dGTP, 7-deaza-dATP, alkyl-pyrimidine nucleotides (including propynyl dUTP) are examples of nucleotides with unconventional bases. Some unconventional sugar modifications include modifications at the 2′-position. For example, ribonucleotides with 2′-OH (i.e., ATP, GTP, CTP, UTP) are unconventional nucleotides for a DNA polymerase. Other sugar analogs and modifications include D-ribosyl, 2′ or 3′ D-deoxyribosyl, 2′,3′-D-dideoxyribosyl, 2′,3′-D-didehydrodideoxyribosyl, 2′ or 3′ alkoxyribosyl, 2′ or 3′ aminoribosyl, 2′ or 3′ mercaptoribosyl, 2′ or 3′ alkothioribosyl, acyclic, carbocyclic or other modified sugar moieties. Additional examples include 2′-PO₄ analogs, which are terminator nucleotides. (See, e.g., U.S. Pat. No. 7,947,817 or other examples described herein). Unconventional linkage nucleotides include phosphorothioate dNTPs ([α-S]dNTPs), 5′-[α-borano]-dNTPs and [α]-methyl-phosphonate dNTPs.

At 214, the selected magnetically-labeled nucleotide precursor is added to the binding area 115.

At 216, sequencing is performed to determine whether the selected magnetically-labeled nucleotide precursor has bound to the polymerase or has been incorporated into the extendable primer. The sequencing step 216 can include multiple sub-steps, as shown in FIG. 7. For example, at sub-step 218 the one or more lines 120 of the apparatus 100 are used to detect a characteristic of the magnetic sensors 105 of the magnetic sensor array 110. As explained above, the characteristic may be, for example, a resistance, a change in resistance, a magnetic field, a change in a magnetic field, a frequency, a change in a frequency, or a noise.

At decision point 220, it is determined whether the detection result indicates that the magnetically-labeled nucleotide precursor has bound to the polymerase or has been incorporated into the extendable primer. For example, the determination may be based on the presence or absence of the characteristic, e.g., if the characteristic is detected, the magnetically-labeled nucleotide precursor is deemed to have bound to the polymerase or to have been incorporated into the extendable primer, and if the characteristic is not detected, the magnetically-labeled nucleotide precursor is deemed not to have bound to the polymerase or have been incorporated into the extendable primer. As another example, the determination may be based on a magnitude or value of the characteristic, e.g., if the magnitude or value is within a specified range, the magnetically-labeled nucleotide precursor is deemed to have bound to the polymerase or have been incorporated into the extendable primer, and if the magnitude or value is not within the specified range, the magnetically-labeled nucleotide precursor is deemed not to have bound to the polymerase or have been incorporated into the extendable primer.

The detection (sub-step 218) and determination (decision point 220) may use or rely on all or fewer than all of the magnetic sensors 105 in the magnetic sensor array 110. The determination of whether the characteristic is present or absent, or the value of the characteristic (decision point 220), may be based on aggregating, averaging, or otherwise processing the detection results (sub-step 218) from some or all of the magnetic sensors 105 in the magnetic sensor array 110.

If, at decision point 220, it is determined that the magnetically-labeled nucleotide precursor has bound to the polymerase or has been incorporated into the extendable primer, then at step 222, an indication of a complementary base of the magnetically-labeled nucleotide precursor is recorded in a record of the nucleic acid sequence of the nucleic acid strand.

In some embodiments, the magnetically-labeled nucleotide precursor is nonextendable by the nucleic acid polymerase, and, therefore, after detecting the characteristic, the magnetic label must be removed to render the magnetically-labeled nucleotide precursor extendable by the nucleic acid polymerase. In some embodiments, a moiety of the first magnetically-labeled nucleotide precursor is not extendable by the nucleic acid polymerase, and the moiety of the first magnetically-labeled nucleotide precursor is rendered extendable by chemical cleavage. As illustrated in FIG. 7, if additional sequencing cycles are to be performed (the “No” path of the decision point 224), the magnetic label is removed at 226 using any suitable means (e.g., chemically, enzymatically, or by other means).

After the magnetic label has been removed at 226, another magnetically-labeled nucleotide precursor is selected at 228. The newly-selected magnetically-labeled nucleotide precursor, which may be the same as or different from the one used in the just-completed cycle, is then added to the binding area 115 at step 214, and the sequencing step 216 is performed again to determine whether the newly-selected magnetically-labeled nucleotide precursor has bound to the polymerase or has been incorporated into the extendible primer.

If, at decision point 220, it is determined that the magnetically-labeled nucleotide precursor has not bound to the polymerase and has not been incorporated into the extendable primer, the method moves to step 228, where another magnetically-labeled nucleotide precursor is selected. In this case, because the previously-tried magnetically-labeled nucleotide precursor was not a match, the selected magnetically-labeled nucleotide precursor should be different from the one used in the just-completed cycle.

Although FIG. 7 shows a single optional wash step 212 occurring between steps 210 and 214, it is to be understood that additional wash steps may be included in the method. For example, the binding area(s) 115 may be washed between steps 228 and 214 or after step 226 (e.g., to substantially remove the previously-introduced magnetically-labeled nucleotide precursor and any magnetic labels removed in step 226). At 230, the method 200 ends.

It is to be understood that after some number of sequencing cycles, it may be desirable or necessary to perform step 210 to add additional molecules of the nucleic acid polymerase to the binding area(s) 115 to replenish the polymerase.

FIG. 7, discussed above, illustrates an embodiment in which magnetically-labeled nucleotide precursors are introduced one at a time. In other embodiments, multiple nucleotide precursors (e.g., two, three, or four nucleotide precursors) are introduced at substantially the same time. In such embodiments, different magnetic labels are used for different nucleotide precursors that are introduced at substantially the same time. Each of the introduced precursor's magnetic label has a different magnetic property that enables the magnetic sensors 105 to distinguish between the different magnetic labels used for the different nucleotide precursors that are introduced at substantially the same time.

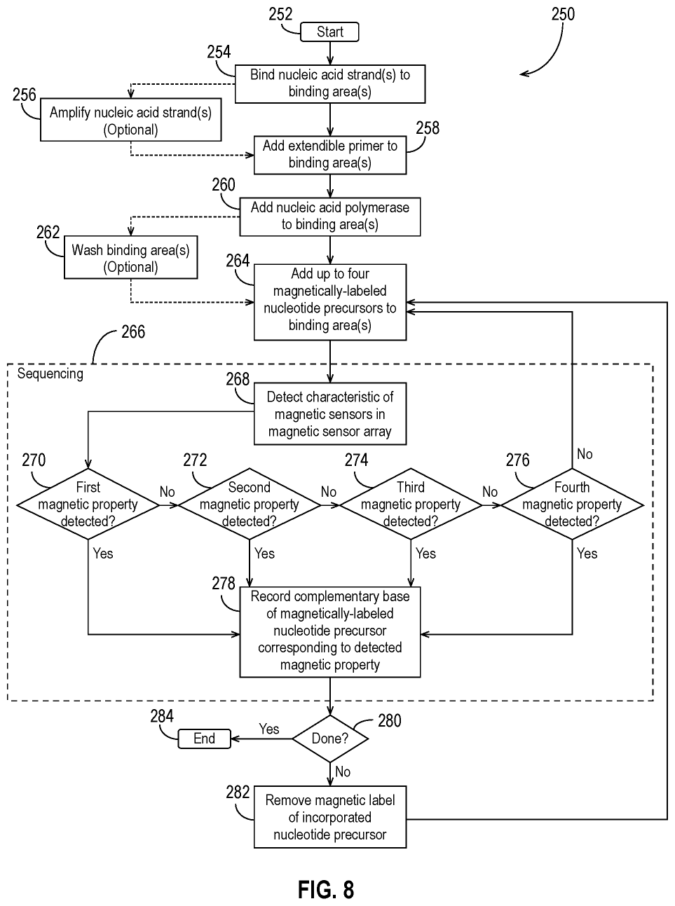

FIG. 8 illustrates an embodiment of a method 250 in which multiple nucleotide precursors are introduced substantially simultaneously to the apparatus 100 or another apparatus that uses magnetic sensors and magnetic labels for detection. For illustration purposes FIG. 8 shows four nucleotide precursors introduced at substantially the same time, but it is to be understood that the disclosed method can be used to test for more or fewer than four nucleotide precursors.

At 252, the method 250 begins. Steps 254, 256, 258, 260, and 262 are the same as steps 204, 206, 208, 210, and 212 shown and described in the context of FIG. 7. That description is not repeated here.

At step 264, up to four magnetically-labeled nucleotide precursors are added to the binding area(s) 115 of the apparatus 100. Each of the added magnetically-labeled nucleotide precursors is labeled with a different magnetic label so that the magnetic sensors 105 can distinguish between the different magnetically-labeled nucleotide precursors. Specifically, each of the magnetic labels has a different and distinguishable magnetic property (e.g., a first magnetic label used for the first magnetically-labeled nucleotide precursor has a first magnetic property, the second magnetic label used for the second magnetically-labeled nucleotide precursor has a second magnetic property, etc.).

At 266, sequencing is performed to determine which of the added magnetically-labeled nucleotide precursors has bound to the polymerase or incorporated into the extendable primer. The sequencing step 266 can include multiple sub-steps, as shown in FIG. 8. For example, in the method 250 illustrated in FIG. 8, at sub-step 268 the one or more lines 120 of the apparatus 100 are used to detect a characteristic of the magnetic sensors 105 of the magnetic sensor array 110, where the characteristic identifies the magnetic property of the incorporated magnetically-labeled nucleotide precursor. As explained above, the characteristic may be, for example, a resistance, a change in resistance, a magnetic field, a change in a magnetic field, a frequency, a change in a frequency, or a noise.

At decision point 270, it is determined whether a first magnetic property has been detected, where the first magnetic property indicates that the first magnetically-labeled nucleotide precursor has bound to the polymerase or has been incorporated into the extendable primer. The determination may be based, for example, on the presence or absence of the first magnetic property, e.g., if the first magnetic property is detected, the first magnetically-labeled nucleotide precursor is deemed to have bound to the polymerase or have incorporated into the extendable primer, and if the first magnetic property is not detected, the first magnetically-labeled nucleotide precursor is deemed not to have bound to the polymerase or incorporated into the extendable primer. As another example, the determination may be based on a magnitude or value of the first magnetic property, e.g., if the magnitude or value is within a specified range, the first magnetically-labeled nucleotide precursor is deemed to have bound to the polymerase or incorporated into the extendable primer, and if the magnitude or value is not within the specified range, the first magnetically-labeled nucleotide precursor is deemed not to have bound to the polymerase or incorporated into the extendable primer.

If it is determined at decision point 270 that the first magnetic property has been detected, the method moves to step 278, where a complementary based of the first magnetically-labeled nucleotide precursor is recorded in a record of the nucleic acid sequence of the nucleic acid strand.

If, at decision point 270, it is determined that the first magnetic property has not been detected, the method 250 moves to decision point 272, at which it is determined whether a second magnetic property has been detected, where the second magnetic property indicates that the second magnetically-labeled nucleotide precursor has bound to the polymerase or incorporated into the extendable primer. The determination may be made in any the ways described above for the determination of the first magnetic property. If it is determined at decision point 272 that the second magnetic property has been detected, the method moves to step 278, where a complementary based of the second magnetically-labeled nucleotide precursor is recorded in a record of the nucleic acid sequence of the nucleic acid strand.

If, at decision point 272, it is determined that the second magnetic property has not been detected, the method 250 moves to decision point 274, at which it is determined whether a third magnetic property has been detected, where the third magnetic property indicates that the third magnetically-labeled nucleotide precursor has bound to the polymerase or incorporated into the extendable primer. The determination may be made in any the ways described above for the determination of the first magnetic property. If it is determined at decision point 274 that the third magnetic property has been detected, the method moves to step 278, where a complementary based of the third magnetically-labeled nucleotide precursor is recorded in a record of the nucleic acid sequence of the nucleic acid strand.

Finally, if, at decision point 274, it is determined that the third magnetic property has not been detected, the method 250 moves to decision point 276, at which it is determined whether a fourth magnetic property has been detected, where the fourth magnetic property indicates that the fourth magnetically-labeled nucleotide precursor has bound to the polymerase or incorporated into the extendable primer. The determination may be made in any the ways described above for the determination of the first magnetic property. If it is determined at decision point 276 that the fourth magnetic property has been detected, the method moves to step 278, where a complementary based of the third magnetically-labeled nucleotide precursor is recorded in a record of the nucleic acid sequence of the nucleic acid strand. If, at decision point 276, it is determined that the fourth magnetic property has not been detected, the method 250 moves back to step 264.

The detection (sub-step 268) and determinations (decision points 270, 272, 274, and 276) may use or rely on all or fewer than all of the magnetic sensors 105 in the magnetic sensor array 110. The determination of whether a particular magnetic property is present or absent, or the value of the characteristic, may be based on aggregating, averaging, or otherwise processing the detection results (sub-step 268) from some or all of the magnetic sensors 105 in the magnetic sensor array 110.

In the embodiment illustrated in FIG. 8, the determination of which of the added magnetically-labeled nucleotide precursors has bound to the polymerase or has been incorporated into the extendable primer is the result of a separate “yes/no” determination for each of the candidate magnetically-labeled nucleotide precursors. It is to be appreciated that the determination can alternatively be made in a single step, such as, for example, by comparing a value of the detected characteristic to a key. For example, the key can indicate that if the characteristic detected by the magnetic sensors 105 has a value in a first range, a first magnetically-labeled nucleotide precursors has bound to the polymerase or incorporated into the extendable primer; if the characteristic detected by the magnetic sensors 105 has a value in a second range, a second magnetically-labeled nucleotide precursors has bound to the polymerase or incorporated into the extendable primer; if the characteristic detected by the magnetic sensors 105 has a value in a third range, a third magnetically-labeled nucleotide precursors has bound to the polymerase or incorporated into the extendable primer; and if the characteristic detected by the magnetic sensors 105 has a value in a fourth range, a fourth magnetically-labeled nucleotide precursors has bound to the polymerase or incorporated into the extendable primer. The value of the characteristic may be based on aggregating, averaging, or otherwise processing the detection results (sub-step 268) from some or all of the magnetic sensors 105 in the magnetic sensor array 110.

As explained above, in some embodiments, the magnetically-labeled nucleotide precursor is nonextendable by the nucleic acid polymerase, and, therefore, after detecting the characteristic, the magnetic label must be removed to render the magnetically-labeled nucleotide precursor extendable by the nucleic acid polymerase. In some embodiments, a moiety of the first magnetically-labeled nucleotide precursor is not extendable by the nucleic acid polymerase, and the moiety of the first magnetically-labeled nucleotide precursor is rendered extendable by chemical cleavage. In embodiments in which the magnetically-labeled nucleotide precursor is nonextendable by the nucleic acid polymerase, after the record of the nucleic acid sequence of the nucleic acid strand has, at step 278, been augmented (or begun), at decision point 280 it is determined whether additional sequencing cycles are to be performed. If so (the “No” branch of decision point 280), the magnetic label of the incorporated nucleotide precursor is removed. The magnetic label may be removed chemically, enzymatically, or by other means known in the art, and the method 250 proceeds to step 264, where up to four magnetically-labeled nucleotide precursors are added to the binding area 115 (potentially after performing a washing step similar or identical to the illustrated step 262). The sequencing step 266 is then performed again to identify the next magnetically-labeled nucleotide precursor to bind to the polymerase.

If, at decision point 280, it is determined that no additional sequencing cycles are to be performed (the “Yes” branch of decision point 280), the method 250 ends at 284.

In the foregoing description and in the accompanying drawings, specific terminology has been set forth to provide a thorough understanding of the disclosed embodiments. In some instances, the terminology or drawings may imply specific details that are not required to practice the invention.

To avoid obscuring the present disclosure unnecessarily, well-known components are shown in block diagram form and/or are not discussed in detail or, in some cases, at all.

Unless otherwise specifically defined herein, all terms are to be given their broadest possible interpretation, including meanings implied from the specification and drawings and meanings understood by those skilled in the art and/or as defined in dictionaries, treatises, etc. As set forth explicitly herein, some terms may not comport with their ordinary or customary meanings.

As used in the specification and the appended claims, the singular forms “a,” “an” and “the” do not exclude plural referents unless otherwise specified. The word “or” is to be interpreted as inclusive unless otherwise specified. Thus, the phrase “A or B” is to be interpreted as meaning all of the following: “both A and B,” “A but not B,” and “B but not A.” Any use of “and/or” herein does not mean that the word “or” alone connotes exclusivity.

As used in the specification and the appended claims, phrases of the form “at least one of A, B, and C,” “at least one of A, B, or C,” “one or more of A, B, or C,” and “one or more of A, B, and C” are interchangeable, and each encompasses all of the following meanings: “A only,” “B only,” “C only,” “A and B but not C,” “A and C but not B,” “B and C but not A,” and “all of A, B, and C.”

To the extent that the terms “include(s),” “having,” “has,” “with,” and variants thereof are used in the detailed description or the claims, such terms are intended to be inclusive in a manner similar to the term “comprising,” i.e., meaning “including but not limited to.” The terms “exemplary” and “embodiment” are used to express examples, not preferences or requirements. The term “coupled” is used herein to express a direct connection/attachment as well as a connection/attachment through one or more intervening elements or structures.

The terms “over,” “under,” “between,” and “on” are used herein refer to a relative position of one feature with respect to other features. For example, one feature disposed “over” or “under” another feature may be directly in contact with the other feature or may have intervening material. Moreover, one feature disposed “between” two features may be directly in contact with the two features or may have one or more intervening features or materials. In contrast, a first feature “on” a second feature is in contact with that second feature.

The terms “substantially” and “approximately” are used to describe a structure, configuration, dimension, etc. that is largely or nearly as stated, but, due to manufacturing tolerances and the like, may in practice result in a situation in which the structure, configuration, dimension, etc. is not always or necessarily precisely as stated. For example, describing two lengths as “substantially equal” or “approximately equal” means that the two lengths are the same for all practical purposes, but they may not (and need not) be precisely equal at sufficiently small scales. As another example, a structure that is “substantially vertical” or “approximately vertical” would be considered to be vertical for all practical purposes, even if it is not precisely at 90 degrees relative to horizontal.

The drawings are not necessarily to scale, and the dimensions, shapes, and sizes of the features may differ substantially from how they are depicted in the drawings.

Although specific embodiments have been disclosed, it will be evident that various modifications and changes may be made thereto without departing from the broader spirit and scope of the disclosure. For example, features or aspects of any of the embodiments may be applied, at least where practicable, in combination with any other of the embodiments or in place of counterpart features or aspects thereof. Accordingly, the specification and drawings are to be regarded in an illustrative rather than a restrictive sense.

1. An apparatus for nucleic acid sequencing, the apparatus comprising:
a plurality of magnetic sensors;
a plurality of binding areas disposed above the plurality of magnetic sensors, each binding area of the plurality of binding areas for holding fluid; and
at least one line for detecting a characteristic of at least a first magnetic sensor of the plurality of magnetic sensors, the characteristic indicating presence or absence of one or more magnetic nanoparticles coupled to a first binding area associated with the first magnetic sensor,
wherein the at least one line includes a first line disposed above a top surface of the first magnetic sensor, and wherein the first binding area is located within a trench in the first line, the trench being above the top surface of the first magnetic sensor.

2. The apparatus recited in claim 1, wherein the first magnetic sensor comprises a magnetoresistive device comprising a pinned layer, a free layer, and a barrier layer disposed between the pinned layer and the free layer.

3. (canceled)

4. The apparatus recited in claim 2, wherein, in the absence of the one or more magnetic nanoparticles coupled to the first binding area, a magnetic moment of the pinned layer is approximately 90 degrees from a magnetic moment of the free layer.

5. (canceled)

6. The apparatus recited in claim 1, wherein a lateral dimension of the first magnetic sensor is between approximately 10 nanometer and approximately 1 micrometer.

7. The apparatus recited in claim 1, further comprising sensing circuitry coupled to the plurality of magnetic sensors via the at least one line, wherein the sensing circuitry is configured to:
apply a current to the at least one line to detect the characteristic of the first magnetic sensor.

8. The apparatus recited in claim 1, wherein the characteristic comprises a magnetic field or a resistance, a change in magnetic field, or a change in resistance.

9. (canceled)

10. The apparatus recited in claim 7, wherein the sensing circuitry comprises a magnetic oscillator, and wherein the characteristic comprises a frequency of a signal associated with or generated by the magnetic oscillator.

11. The apparatus recited in claim 1, wherein the characteristic comprises a noise level.

12. The apparatus recited in claim 1, further comprising an insulating material disposed between the plurality of magnetic sensors and the plurality of binding areas.

13. The apparatus recited in claim 12, wherein the insulating material comprises at least one of silicon dioxide, aluminum oxide, or silicon nitride.

14. The apparatus recited in claim 12, wherein the insulating material comprises at least one of an oxide or a nitride.

15. The apparatus recited in claim 12, wherein a thickness of the insulating material between a top of the first magnetic sensor and the first binding area is between approximately 3 nanometers and approximately 20 nanometers.

16. (canceled)

17. The apparatus recited in claim 1, wherein the plurality of magnetic sensors is arranged in a rectangular array, and wherein the at least one line further includes a second line, wherein the second line is disposed below the first magnetic sensor.

18. (canceled)

19. The apparatus recited in claim 17, wherein the first line is coupled to a row of the rectangular array and the second line is coupled to a column of the rectangular array, or vice versa.

20. The apparatus recited in claim 1, wherein the first binding area comprises a structure configured to anchor nucleic acid to the first binding area, wherein the structure comprises a cavity or a ridge.

21. (canceled)

22. A method of sequencing nucleic acid using an apparatus comprising a plurality of magnetic sensors, a plurality of binding areas disposed above the plurality of magnetic sensors, each binding area of the plurality of binding areas for holding fluid, and at least one line for detecting a characteristic of at least a first magnetic sensor of the plurality of magnetic sensors, the at least one line including a first line disposed above a top surface of the first magnetic sensor, a first binding area being located within a trench in the first line, the trench being above the top surface of the first magnetic sensor, the method comprising:
(a) binding a nucleic acid strand to the first binding area;
(b) in one or more rounds of addition, adding, to the first binding area, an extendable primer and nucleic acid polymerase;
(c) adding, to the first binding area, a first nucleotide precursor, the first nucleotide precursor labeled by a first cleavable magnetic label; and
(d) sequencing the nucleic acid strand,
wherein sequencing the nucleic acid strand comprises:
using the at least one line, detecting the characteristic of the first magnetic sensor, the characteristic indicating presence or absence of the first cleavable magnetic label.

23. (canceled)

24. The method recited in claim 22, further comprising amplifying the nucleic acid strand after binding the nucleic acid strand to the first binding area.

25. The method recited in claim 24, wherein, as a result of the amplifying, one or more amplicons are bound to the first binding area.

26. The method recited in claim 22, wherein sequencing the nucleic acid strand further comprises:
in response to a determination that the characteristic indicates the presence of the first cleavable magnetic label coupled to the first binding area, recording a complementary base of the first nucleotide precursor in a record of a nucleic acid sequence of the nucleic acid strand.

27. The method recited in claim 22, wherein the first nucleotide precursor is nonextendable by the nucleic acid polymerase, and further comprising:
after detecting the characteristic, removing the first cleavable magnetic label and rendering the first nucleotide precursor extendable by the nucleic acid polymerase.

28-29. (canceled)

30. The method recited in claim 22, further comprising, after sequencing the nucleic acid strand, removing the first cleavable magnetic label by enzymatic or chemical cleavage.

31. The method recited in claim 22, further comprising:
repeating steps (c) and (d) with a different nucleotide precursor during each repetition, each of the different nucleotide precursors being magnetically labeled.

32. (canceled)

33. The method recited in claim 22, further comprising washing the first binding area before step (c).

34. The method recited in claim 22, wherein the first cleavable magnetic label has a first magnetic property, and wherein the method further comprises:
in the one or more rounds of addition, adding, to the first binding area, a second nucleotide precursor labeled by a second cleavable magnetic label having a second magnetic property.

35. The method recited in claim 34, further comprising:
in the one or more rounds of addition, adding, to the first binding area, a third nucleotide precursor labeled by a third cleavable magnetic label having a third magnetic property, and a fourth nucleotide precursor labeled by a fourth cleavable magnetic label having a fourth magnetic property.

36. The method recited in claim 22, wherein binding the nucleic acid strand to the first binding area comprises:
attaching an adapter to an end of the nucleic acid strand; and
coupling an oligonucleotide to the first binding area, wherein the oligonucleotide is capable of hybridizing to the adapter.

37. The method recited in claim 22, wherein binding the nucleic acid strand to the first binding area comprises covalently bonding the nucleic acid strand to the first binding area or immobilizing the at least one nucleic acid strand via irreversible passive adsorption or affinity between molecules.

38. (canceled)

39. The method recited in claim 22, wherein the first binding area comprises a cavity or a ridge, and wherein binding the nucleic acid strand to the first binding area comprises applying a hydrogel to the cavity or to the ridge.

40. The method recited in claim 22, further comprising:
after step (c), adding, to the first binding area, additional molecules of the nucleic acid polymerase.

41. The method recited in claim 22, wherein the first cleavable magnetic label comprises a magnetic nanoparticle.

42. The method recited in claim 41, wherein the magnetic nanoparticle comprises a molecule, a superparamagnetic nanoparticle, or a ferromagnetic nanoparticle.

43-44. (canceled)

45. The method recited in claim 22, wherein the first nucleotide precursor comprises one of dATP, dGTP, dCTP, dTTP, or equivalents.

46. The method recited in claim 22, wherein the nucleic acid polymerase comprises a Type B polymerase lacking 3′-5′ exonuclease activity, or a thermostable polymerase.

47. (canceled)

48. The method recited in claim 22, wherein using the at least one line comprises applying a current to the at least one line.

49. The method recited in claim 22, wherein the characteristic comprises a magnetic field or a resistance, a frequency of a signal associated with or generated by a magnetic oscillator, a noise level, a change in magnetic field, or a change in resistance.

50-75. (canceled)
